(19) United States
(12) Patent Application Publication
(10) Pub. No.: US 2026/0305985 A1
(43) Pub. Date: Oct. 8, 2026

(54) MONITORING A CHEMICAL ANALYTE IN A PATIENT'S BLOOD USING AN UNDERLID DETECTOR

(71) Applicant: Verily Life Sciences LLC, Dallas, TX (US)

(72) Inventor: Timothy Stowe, South San Francisco, CA (US)

(21) Appl. No.: 19/483,081

(22) PCT Filed: May 2, 2024
(86) PCT No.: PCT/US2024/027373

§ 371 Date: Nov. 10, 2025

Related U.S. Application Data
(60) Provisional application No. 63/465,660, filed on May 11, 2023.

(51) Int. Cl. A61B 5/145; A61B 5/00; A61B 5/1455

(52) U.S. Cl. CPC A61B 5/14532; A61B 5/0002; A61B 5/1455

(57) ABSTRACT

A chemical analyte can be monitored in the blood of a patient's palpebral conjunctiva. A device, including a mounting substrate configured to hold a microcircuit and to mount optoelectronic components, like one or more micro light emitting diodes (LEDs) and a micro photodiode detector adjacent to the one or more LEDs, can be inserted beneath a lower eyelid of the patient and above an eye. The mounting substrate and the optoelectronic components can be covered and protected by a casing including an infrared transparent encapsulation material, with a surface of the infrared transparent encapsulation material having hydrophobic surface properties in a region above the micro LEDs and the photodiode detector. The micro LEDs can emit short wave infrared light with wavelengths from 1300 nm to 1700 nm toward the palpebral conjunctiva. The photodetector can detect light reflected from the palpebral conjunctiva to monitor the chemical analyte.

### CROSS-REFERENCE TO RELATED APPLICATIONS

This application claims the benefit of U.S. Provisional Application Ser. No. 63/465,660, filed May 11, 2023, entitled “MONITORING A CHEMICAL ANALYTE IN A PATIENT'S BLOOD USING AN UNDERLID DETECTOR”. The entirety of this application is incorporated by reference for all purposes.

### TECHNICAL FIELD

The present disclosure relates to monitoring a chemical analyte in a patient's blood, and, more specifically, to systems and methods for detecting and monitoring a chemical analyte in a patient's blood using a non-invasive detector placed under a patient's lower eyelid (e.g., an underlid detector).

### BACKGROUND

Approximately 463 million people worldwide suffer from diabetes. Type 2 diabetes makes up approximately 90% of those cases. Improperly managed diabetes can lead to significant health deterioration and even death in some cases. While diabetes can be managed with monitoring and timely treatment, medical expenses related to diabetes can be quite high and monitoring devices can be expensive and difficult to use. The key to diabetes management is keeping the patient's blood sugar in range because patients who have well-managed blood sugar levels are less likely to suffer from complications and those complications that do occur tend to be less severe. Traditional devices used for diabetes management such as glucometers and test strips, insulin patches, pumps, and pens, and newer devices like continuous glucose monitors (CGMs) have improved the quality of life for diabetics but are very expensive to the healthcare system and often have inherent patient-related compliance issues. Current diabetes management devices (both traditional and recently developed) tend to require chemical detection directly from a blood sample and/or skin penetration.

A goal of diabetes management is an accurate non-invasive solution for glucose detection (e.g., using devices that can be worn around the wrist or finger for optical glucose detection) for type I or type II diabetes management. Currently wearable non-invasive glucose sensing devices use some form of optical detection but have not reached an acceptable level of accuracy. Optical detection allows for a long sensor life at a lower cost and requires no skin penetration. Generally, optical detection approaches use 900-1200 nm wavelengths to reach vessels buried deep under the skin. However, due to the depth of the blood vessels being measured from the finger or the wrist, the signals representative of glucose are muddied by competing signals (e.g., due to hydration, BMI, blood albumin, and the like) that are also inadvertently sensed. Thus, the accuracy of optically detecting analytes such as glucose is limited because these measurements are sensitive to skin color, BMI, age, hydration, gender, temperature, intervening materials, and instrumentation shift due to relative skin motion.

Since the wrist and finger have proven so difficult to use, researchers have investigated other locations that may not have these limitations. In the late 1990's some research on non-invasive glucose detection was directed towards the mucosal tissue under a patient's lips. The mucosal tissue has micro-blood vessels much closer to the surface of the skin than on the wrist or finger. Diffuse reflectance spectroscopy was done from 900-2500 nm by various groups but it was found the glucose signal strength, though pronounced, was subject to drift which can occur from varying amounts of saliva that contribute to the background water signal and from the absence or presence of some positive pressure against the skin to assist in intimate contact with the tissue. While glucose detection measurements were notably pronounced and accurately tracked with blood glucose when very stringent protocols were implemented, the results were limited by practical issues such as mouth motion during speech and the lack of miniaturized sensors and emitters. Thus, these practical spectral detection issues and user protocol requirements limited the applicability of the research and required substantial consideration as well as scientific spectral analysis for a practical non-invasive sensor.

### SUMMARY

A non-invasive (and non-implantable) chemical analyte sensing device need not be limited to a finger or wrist wearable or a device placed in a person's mouth. Instead, a non-invasive and non-implantable chemical analyte sensing can be an insertable and removable from under a patient's lower eyelid (e.g., underlid device). Such an underlid device can be used for improved monitoring of one or more chemical analytes in a patient's blood (e.g., glucose) because vessels there are generally located less than 100 μm under the skin with a high structural uniformity from person to person and are more uniform than other mucosal tissue areas. Additionally, the underlid area can provide a natural pocket to hold a sensor in place and apply a small amount of pressure to ensure the device can stay at least partially pressed against the tissue site to be measured. Furthermore, a higher wavelength of light in the SWIR spectrum can be used so that the signal indicative of the analyte is generally not buried in competing signals through the use of newly developed micro-emitters and detectors capable of operating with these higher infrared wavelengths.

In one aspect, the present disclosure includes such a non-implantable, non-invasive device that can be configured to monitor a chemical analyte in a patient's blood. A mounting substrate can be configured to hold at least one microcircuit and optoelectronic components (e.g., light sources like one or more micro light emitting diodes LEDs) configured to emit short wave infrared (SWIR) light with peak wavelengths falling between 900 nm to 1700 nm toward a palpebral conjunctiva of the patient; and a micro photodiode detector positioned adjacent to the one or more micro LEDs and configured to detect reflected light from the palpebral conjunctiva of the patient after the short wave infrared light is emitted by the one or more micro LEDs. A soft curved semi-flexible casing can be configured to fit between the lower eyelid and an eyeball of the patient and can encapsulate the mounting substrate and the optoelectronic components. The casing can include an infrared transparent material which is bio compatible. This could, for example, be a thermoplastic overmold material known as Polystyrene-block-Isobutylene-block-Styrene or polydimethylsiloxane or an infrared transparent bio-compatible moisture ingress protection glop top epoxy. A portion of the infrared transparent material above at least one or more micro LEDs and the micro photodiode detector can include hydrophobic surface properties. The device can be configured to be inserted under a lower eyelid of the patient and over the outer white sclera surface of the eyeball such that micro LED optical signals are directed towards the palpebral conjunctiva.

A portion of the infrared transparent material above at least the one or more micro LEDs and the micro photodiode detector can include hydrophobic surface properties for inhibiting tear film formation between the underlid device and the palpebral conjunctiva. Furthermore, the surface including the hydrophobic surface properties can be infused with an eyesafe monograph of biocompatible oil that can further suppresses water-based tear film formation and form an IR transparent oil based meniscus between the sclera and the palpebral conjunctiva.

In another aspect, the present disclosure includes a method for monitoring a chemical analyte in the blood of a patient's palpebral conjunctiva. The device can include: a mounting substrate configured to hold a microcircuit and to mount optoelectronic components, wherein the optoelectronic components can include: light sources, like one or more micro light emitting diodes (LEDs); and a micro photodiode detector, adjacent to the one or more LEDs. The mounting substrate and the optoelectronic components can be covered and protected by an infrared transparent encapsulation material, with a surface of the infrared transparent encapsulation material having hydrophobic surface properties in a region above the one or more micro LEDs and the photodiode detector. The one or more micro LEDs can emit short wave infrared light with peak output wavelengths ranging from 900 nm to 1700 nm toward the palpebral conjunctiva; and the photodetector can detect light reflected off the palpebral conjunctiva after receiving the short wave infrared light emitted by the one or more micro LEDs.

### BRIEF DESCRIPTION OF THE DRAWINGS

The foregoing and other features of the present disclosure will become apparent to those skilled in the art to which the present disclosure relates upon reading the following description with reference to the accompanying drawings, in which:

FIG. 1 is an illustration of an example system with a non-invasive underlid chemical analyte detector that can communicate with a patient's wireless device;

FIG. 2 shows examples of shapes and compositions of the example non-invasive underlid chemical analyte detector of FIG. 1;

FIG. 3 is an illustration of the placement of the example non-invasive underlid chemical analyte detector of FIG. 1;

FIGS. 4 and 5 are diagrams illustrating the anatomy of the conjunctiva around the eye where the example non-invasive underlid chemical analyte detector of FIG. 1 is placed;

FIG. 6 is a diagram showing a front view of the example non-invasive underlid chemical analyte detector of FIG. 3;

FIG. 7 is a diagram showing a front view of the example non-invasive underlid chemical analyte detector of FIG. 6 with an example of the microcircuit and the optoelectronic components;

FIGS. 8-9 are diagrams showing a front view of the example non-invasive underlid chemical analyte detector of FIG. 7 with different configurations of the wireless antenna;

FIG. 10 is a diagram showing a side view of the non-invasive underlid chemical analyte detector of FIG. 6;

FIG. 11 is a diagram showing a side view of the non-invasive underlid chemical analyte detector of FIG. 6 in the operation of detecting glucose in a patient's blood; and

FIG. 12-15 are process flow diagrams of example methods for monitoring a chemical analyte in a patient's blood using a non-invasive underlid detector.

### DETAILED DESCRIPTION

### I. Definitions

Unless otherwise defined, all technical terms used herein have the same meaning as commonly understood by one of ordinary skill in the art to which the present disclosure pertains.

As used herein, the singular forms “a,” “an,” and “the” can also include the plural forms, unless the context clearly indicates otherwise.

As used herein, the terms “comprises” and/or “comprising,” can specify the presence of stated features, steps, operations, elements, and/or components, but do not preclude the presence or addition of one or more other features, steps, operations, elements, components, and/or groups.

As used herein, the term “and/or” can include any and all combinations of one or more of the associated listed items.

As used herein, the terms “first,” “second,” etc. should not limit the elements being described by these terms. These terms are only used to distinguish one element from another. Thus, a “first” element discussed below could also be termed a “second” element without departing from the teachings of the present disclosure. The sequence of operations (or acts/steps) is not limited to the order presented in the claims or figures unless specifically indicated otherwise.

As used herein, the term “palpebral conjunctiva” refers to a clear (thin, mucous) membrane that lines the inside of a patient's eyelids. In addition to containing fibrous tissue and lymphatic channels, the palpebral conjunctiva includes a dense array of vascularized microvessels, generally supplied by the ophthalmic artery, which branches off from the main external carotid artery. Many of these microvessels are less than 100 μm from the surface of the palpebral conjunctiva.

As used herein, the term “underlid” refers to something (e.g., a device) that is positioned beneath a patient's eyelid in the space between the eyelid and an eye. While the term “underlid” generally refers to under a lower eyelid, the term “underlid” can also refer to under an upper eyelid, and above the eye.

As used herein, the term “chemical analyte” refers to a chemical substance or constituent that is of interest and being monitored and/or analyzed. A non-limiting example of a chemical analyte is glucose. Other examples of chemical analytes include, but are not limited to blood lipids like cholesterol, potassium, iron, inorganic phosphorus, magnesium, lactate dehydrogenase (LDH), lactate, insulin, parathyroid hormone, osteocalcin, adrenocorticotropic hormone, or the like.

As used herein, the term “microcircuit” refers to one or more integrated electrical circuits that implement several components or their equivalents and is sized on a micro-or nano-scale. For example, the microcircuit can be embodied on a mounting substrate, like a flexible printed circuit board (PCB).

As used herein, the term “optoelectronic components” refers to devices and systems that can find, detect, emit, and/or control light. Optoelectronic components can include electrical-to-optical or optical-to-electrical transducers. Non-limiting examples of optoelectronic components can include light emitting diodes (LEDs) (or other light emitters), photodetectors (or other light detectors), and the like. It should be understood that the term “photodetector” encompasses detection of light by photodetectors and detection by additional devices like photodiodes, phototransistors, and the like.

As used herein, the term “infrared transparent encapsulation material” refers to a material that is substantially transparent to at least infrared light (e.g., allows an amount of infrared light to pass through) and can encase one or more objects (such as at least a portion of the mounting substrate and components positioned thereon). A surface of an infrared transparent encapsulation material can have one or more of hydrophobic surface properties in one region and hydrophilic surface properties in another region.

As used herein, the term “hydrophobic surface properties” refers to the properties of a material that repels water and has a low surface energy surface. Water on a material having hydrophobic surface properties will exhibit a high contact angle and bead up.

As used herein, the term “high viscosity” can refer to a fluid that moves sluggishly and resist deformation. Generally, “viscosity” of a fluid is the measure of its resistance to deformation at a given rate and it quantifies the internal frictional forces between adjacent layers of a fluids that are in relative motion. An example of a high viscosity fluid can be an “oil-based coating”, like any medical grade mineral oil and/or a medical grade white petrolatum.

As used herein, the term “near field communication (NFC)” can refer to a set of communication protocols that enable communication between two electronic devices (one comprising at least a transmitter and the other comprising at least a receiver) over distances of 4 cm or less.

As used herein, the terms “patient”, “subject”, “user”, and the like can be used interchangeably and can refer to an animal (e.g., a human) having a condition that can be monitored with an underlid sensing device.

### II. Overview

Successful long term management of blood sugar levels can prevent diabetics from suffering major complications of diabetes and improve patient outcomes when complications do occur. Blood sugar levels can be well-managed with monitoring and treatment when levels become unhealthy. However, not all patients appropriately monitor and/or control their blood sugar levels, with some causes being that monitoring devices can be expensive and difficult for the patients to use and are not always calibrated correctly for all demographics. Thus, improperly controlled diabetes can still lead to significant health deterioration and even death in patients with sub-optimal control of blood sugar levels.

There has been a significant push to make blood sugar monitoring devices that are easier for patients to use. For example, continuous glucose monitors (CGMs) designed to be at least partially inserted under the skin, generally of the stomach or arm, are now commercially available. Although CGM devices can be small, CGM devices are invasive and still require insertion of a needle or sensor under the skin and last about 7-14 days. Non-invasive optical blood glucose detection using a finger or wrist wearable is not yet a uniformly viable commercial solution. One reason being that non-invasive optical-based glucose detection generally uses visible light and/or near infrared (NIR) light limited between wavelengths from 900-1200 nm due to the penetration depth required at such locations and thus an analyte spectral signal suffers from interference from large overlapping spectral signals (e.g., from fat, bone, proteins, melanin, red blood cells, etc.) in addition to dependence on body type. Optical spectroscopy in the visible (400-700 nm) and the near-infrared (700-1000 nm) wavelength range has been used as a non-invasive diagnostic technique for determining the concentrations of various biological analytes. Non-invasive medical devices such as these measure the optical attenuation of light which enables imaging depths up to several millimeters. In this wavelength region, tissue scatter is low and unfortunately the detected signal levels are limited by the effects from hemoglobin absorption and the lack of strong absorption bands for many analytes such as glucose. At shortwave infrared (SWIR) wavelengths (900-1200 nm), hemoglobin absorption is low and several analytes such as blood, water, glucose and lipids have distinct spectral signatures which enhance their detection and characterization. Various analytical techniques such as multivariate calibration, principal component analysis, or machine learning algorithms can be used to extract the analyte concentration from these measurements by non-invasively probing the tissue. Measurements are typically made in the reflectance geometry.

Described herein is a device that can monitor and detect glucose and/or at least one other chemical analyte in a patient's blood (e.g., chemical analytes indicative of high cholesterol, anemia, glaucoma, dry eye disease, or the like) that is smaller than the current smallest CGM, requires no needles, and optically detects blood glucose and/or at least one other chemical analyte directly using optoelectronics. Such a device can be positioned under an eyelid of a patient, does not require implantation, can last longer than current devices, and can be invisible or nearly so to the naked eye. The device can be a non-invasive (and non-implantable) sensing device that can detect glucose and/or other chemical analytes in a patient's blood from under the patient's lower eyelid (e.g., underlid device). The underlid device can optically detect chemical analytes (e.g., glucose or the like) in the blood flowing through microvessels of the patient's palpebral conjunctiva (less than 100 μm under the skin) using short wave infrared light signals (e.g., wavelength 900 nm-1700 nm) detected by reflectance spectroscopy. This detection method works due to the minimal depth of penetration of the light signals needed to measure through the palpebral conjunctiva.

### III. System

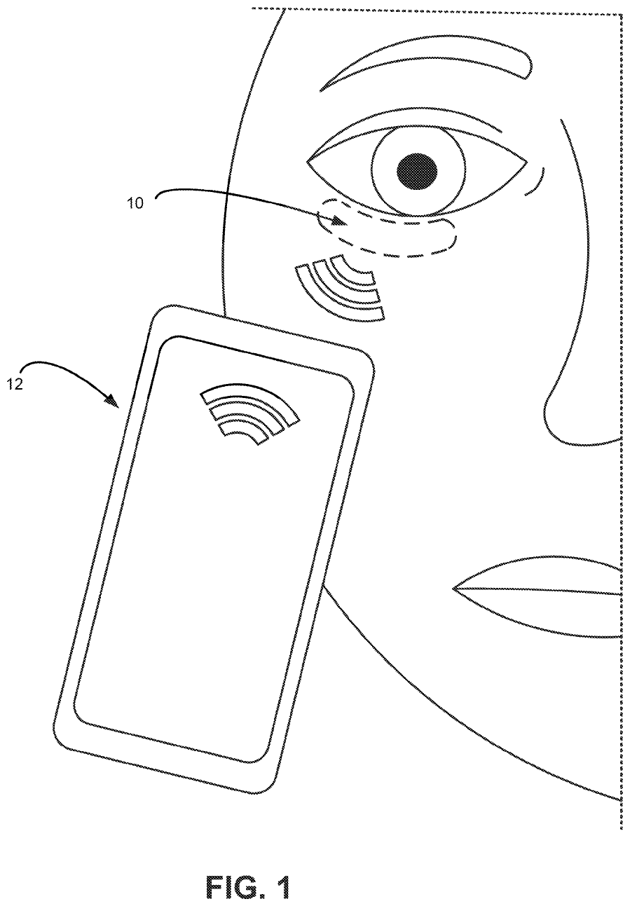

Provided herein is a system (FIG. 1) with a non-invasive underlid chemical analyte detector (also referred to as “the device”, “the non-invasive underlid detector” the “monitoring device”, and the like) 10 that can communicate with a patient's wireless device 12 (also referred to as “the remote device 12”). For example, the patient's wireless device 12 can be a smartphone, tablet, smartwatch, or other type of device capable of wireless communication. In some instance, a wireless device may belong to a medical professional, which can be used to program the device 12 beyond the capabilities of the patient's wireless device 12 The patient's wireless device 12 may be equipped with a plug in boost near field communication (NFC) antenna (e.g., a wire wound coil reader), and the device 10 can have a micro-coil NFC antenna. For example, the device 10 can include a wireless micro-coil antenna, based on NFC standards, that can engage in wireless communication with a remote transponder (e.g., of the patient's wireless device 12) that includes an NFC reader. In some instances, other wireless communication standards, other than NFC (e.g., WIFI or Bluetooth or the like) could be used, additionally and/or alternatively, for data communications and power transfer between the device 10 and the mobile device 12 including the necessary technical components.

The underlid chemical analyte detector 10 can be designed and used to detect and monitor any chemical analyte in a patient's blood. An example of a chemical analyte in the patient's blood is glucose. Other examples of chemical analytes in blood include, but are not limited to cholesterol, potassium, iron, inorganic phosphorus, magnesium, lactate dehydrogenase (LDH), lactate, insulin, parathyroid hormone, osteocalcin, adrenocorticotropic hormone, or the like. The device 10 can be positioned under the lower eyelid of a patient such that the optical components face towards the palpebral conjunctiva to detect for chemical analytes in the blood in blood vessels of the palpebral conjunctiva. The device 10 can be shaped and sized to fit between the eye and the palpebral conjunctiva in a manner comfortable for the patient. The patient's mobile device 12 can be placed in proximity to the underlid chemical analyte detector 10 and engage in NFC, for example, to collect information from, or send information to, the underlid chemical analyte detector positioned under the eyelid of the patient. The underlid chemical analyte detector 10 can communicate with the patient's wireless device 12 to exchange data and/or commands. Additionally or alternatively, the patient's wireless device 12 can transmit a power signal to the device 10 that can be used to power the associated circuitry (e.g., micro battery, etc. in the device). In one instance, the device 10 and/or the patient's wireless device 12 can employ higher permeability ferrite micro coil technology for NFC energy harvesting and NFC 13.56 MHz communications.

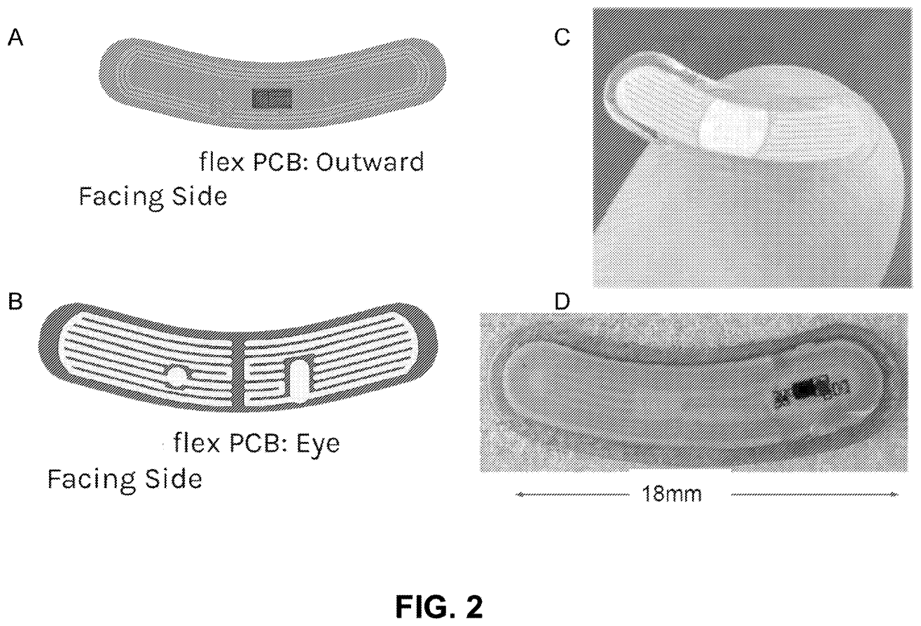

Example illustrations and pictures of components that may be used in the underlid chemical analyte detector 10 are shown in FIG. 2, elements A-D. For example, device 10 can be flexible and designed with rounded edges and curved such that the device can comfortably fit between the eye and eyelid of a patient (e.g., without irritating the eye or the eyelid). The device 10 can be flexible, including a flexible printed circuit board (PCB) and one or more flexible encapsulating materials that are non-harmful to the eye to improve the patient's comfort. As an example, the PCB can include and/or connect an NFC reader to allow for energy harvesting and/or a micro battery to power at least one or more light sources (referred to herein with the example LEDs, but not confined to LEDS) and a photodetector within the device 10. Element A of FIG. 2 shows an outward facing side (e.g., facing towards the eyelid) of an example flexible PCB of an underlid chemical analyte detector including depictions of traces, circuit elements, and optoelectronic components. Element B of FIG. 2, shows an eye facing side of an example flexible PCB of an underlid chemical analyte detector including depictions of the traces, circuit elements, and other components that may not be required on the outward facing side of the detector. FIG. 2, Element C shows an example of the underlid chemical analyte detector shape in 3D in relation to a fingertip to show an example of the size of the device and the angles of the curves of the device. For example, the bottom and top of the device are curved in a kidney or half-moon shaped manner and the device also subtly curves towards the eye with an angle similar to or the same as the curvature of the eye. FIG. 2, Element D shows an example of the underlid chemical analyte detector that is 18 mm in length (however, it should be noted that other lengths are possible between 3 and 20 mm, for example).

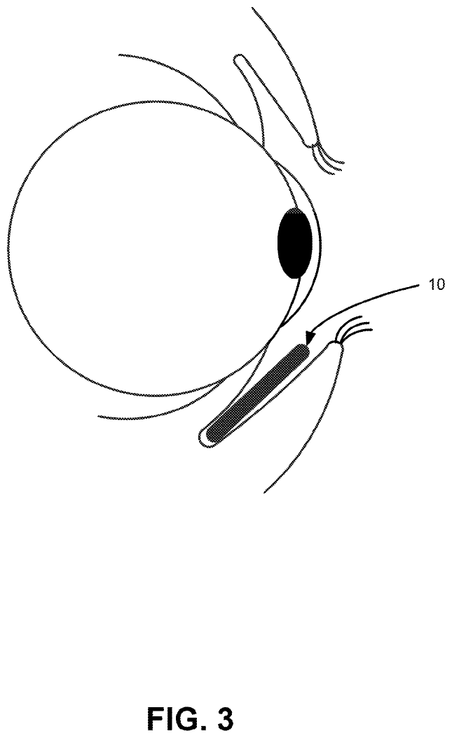

FIG. 3 shows a simplified illustration of a side view of a patient's eye. In FIG. 3 the illustration of the eye includes the eyeball covered by an upper and lower eyelid. Generally, the eyelids are thin structures that include skin, subcutaneous tissue, muscle, fat pads, conjunctiva, nerves, and vessels. The device 10 is positioned under a portion of the lower eyelid within the conjunctiva between a posterior surface of the lower eyelid and the anterior surface of the eyeball (and/or muscles and other biological tissues attached to the eyeball). The device 10 is shown with a pill shaped outline, but may have any shape and or curvature that is comfortable for the patient. Using the examples shown in FIG. 2, the outward facing side (element A) of the device can face the posterior surface of the lower eyelid and the eye-facing side (element B) of the device can face the anterior surface of the eyeball. However, given the present size of micro LEDs and micro infrared detectors, the device may be as small as 3.5 mm long and 1.25 mm in height. It should be understood that the device can have any length between 3 mm to 20 mm and any height between 0.5 mm to 10 mm. (generally in a ratio of about 1 mm height to about 3 mm length). The thickness of the device substrate and components is advantaged to be as thin as possible for comfort, ideally as small as 0.2 mm to no more than 1.5 mm.

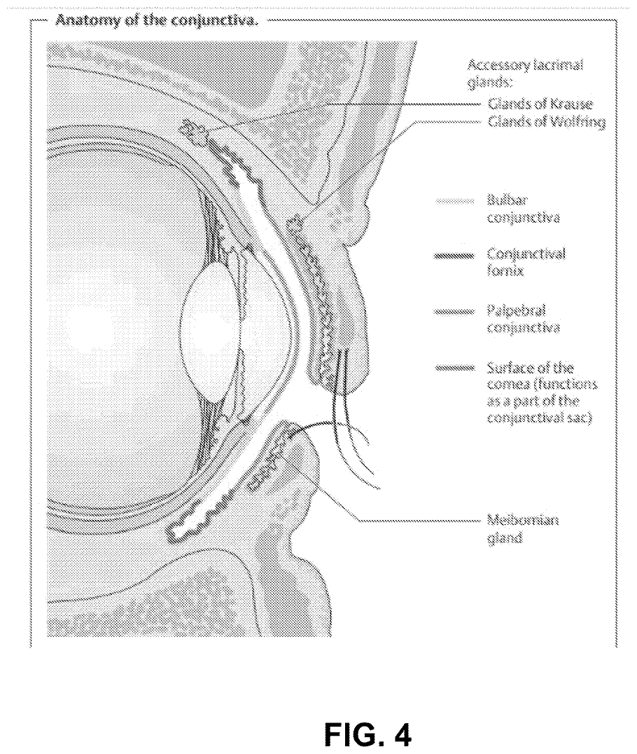

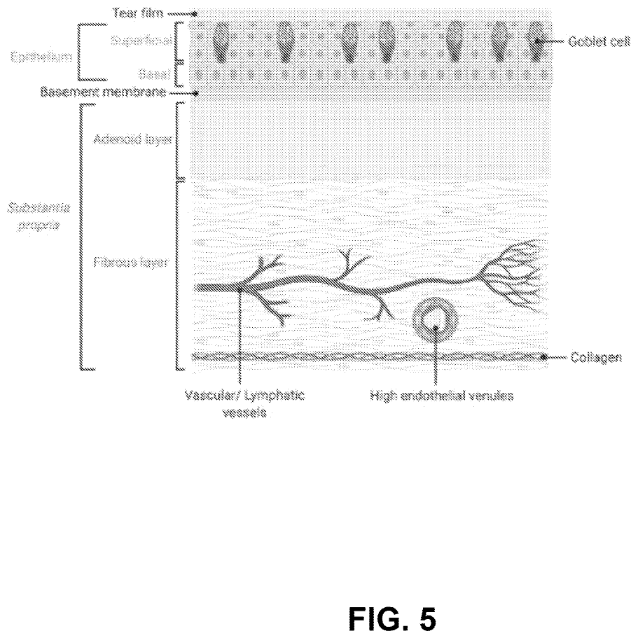

It should be noted that although not shown in the simplified illustration of FIG. 3, the eye conjunctiva is a complex structure with several distinct layers and changes around the eye sac as shown in FIGS. 4-5. The conjunctiva is a fragile yet complex mucosal membrane located anterior to the white sclera, beginning at the corneal limbus and extending to cover the inner surface of the eyelids. The conjunctiva is divided into three distinct regions which are the bulbar, forniceal, and tarsal (or palpebral) parts as shown in FIG. 4. The bulbar conjunctiva covers the surface of the eyeball and forms a clear skin that does not carry many blood vessels and a very thin fibrous layer. The forniceal conjunctiva arches around the superior and inferior cul-de-sacs. The palpebral conjunctiva covers the inner eyelid and adheres firmly to the tarsal plate. Histologically, the palpebral conjunctiva comprises the superficial epithelium, the basal epithelium, the adenoid layer, and the fibrous layer as shown in FIG. 5. The fibrous layer contains a rich thick layer of dense micro-vascular blood vessels. In the palpebral conjunctiva region, the epithelium layer is generally very thin and holds only 2-layers of columnar and flattened basal cells which are stratified columnar in structure and typically about 35-40 micrometers thick. The adenoid layer in the palpebral is similar in thickness to the epithelium layer whereas the fibrous layer containing micro blood vessels in the palpebral region can be very dense and on the order of 160 micrometers thick. Thus, unlike other areas of the body, these blood vessels in the palpebral conjunctiva are very close to the surface of the epithelium and extremely dense. Furthermore, these particular micro-capillary blood vessels in the fibrous layer are generally extremely uniform in comparison to other known blood vessels across a wide variety of people and body types. Therefore, device 10 can be positioned near/against the palpebral conjunctiva with minimal, little, or none of the typical skin interferences seen at other skin locations. Additionally, the conjunctival sac can provide a slight pressure against the palpebral tissue, even when a patient blinks, thus providing pressure on the device 10 that can keep the device in a stable arrangement against the tissue to be measured. Accordingly, the palpebral conjunctiva is an ideal location for shallow detection of analytes in the blood.

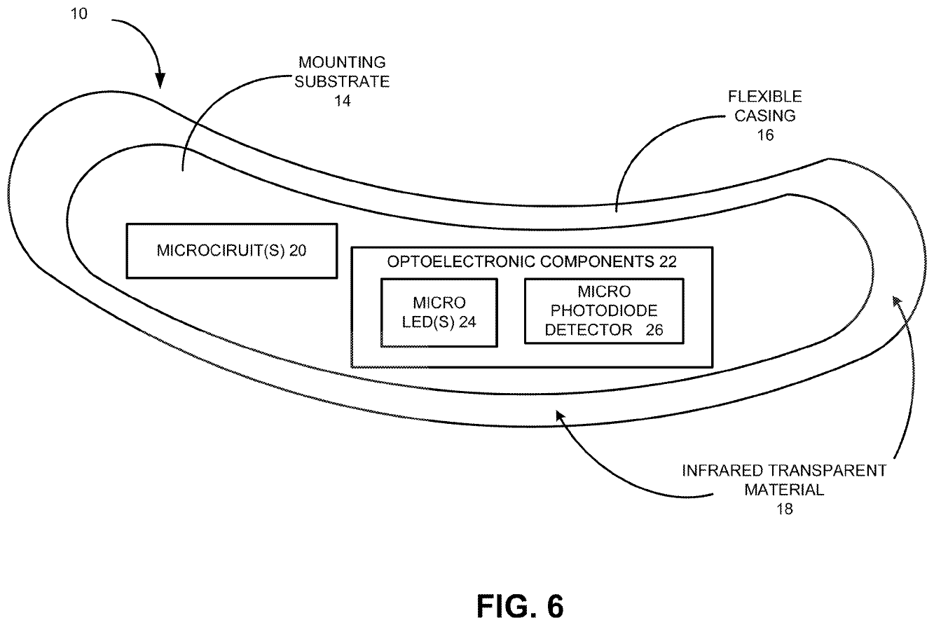

Referring to FIG. 6, illustrated is a schematic diagram showing a front view of the example non-invasive underlid chemical analyte detector (device 10) configured to be inserted under a patient's lower eyelid (between the lower eyelid and the eye). The device 10 can include a mounting substrate 14 that can hold and/or connect electrical components encapsulated in flexible casing 16. The mounting substrate 14 can be the flexible PCB depicted in FIG. 2. For example, the mounting substrate 14 can be an active flex PCB that can bend with a radius of curvature of the patient's eyeball. The mounting substrate 14 can hold at least one microcircuit (illustrated as microcircuit(s) 20) and optoelectronic components 22. As mentioned, the mounting substrate 14 can be encapsulated by a flexible casing 16 such that the encapsulation can be complete around the mounting substrate and the components on the mounting substrate. The flexible casing 16 can include one or more materials that are at least substantially non-immunogenic and non-cytotoxic when inserted between the lower eyelid and an eyeball of the patient (generally without penetrating the eyeball). The flexible casing 16 can include an infrared transparent material 18. A portion of the infrared transparent material 18 at least above the optoelectronic components 22 (between the optoelectronic components 22 and the eyelid, not shown in FIG. 6) can exhibit hydrophobic surface properties to repel tears and other fluids to control the amount of water absorption during the optical detection (discussed more below). The remainder of the infrared transparent material 18 can have hydrophilic surface properties to attract the tears and other watery fluids away from the area between the optoelectronic components and the eyelid.

Additionally, at least a portion of the casing above the optoelectronic components 22 can also be covered with a high viscosity oil-based coating (e.g., medical grade mineral oil, a medical grade white petrolatum, or the like) to further repel water from that area and/or suppress the tear layer. The high viscosity oil-based coating can also lubricate the device 10 to improve comfort of the patient using the device. The hydrophobic material coatings of the device 10 may not be enough on their own to entirely suppress the tear layer which has complex natural surfactants (mucins, proteins, or the like), which may still adhere to the hydrophobic coating, allowing for a tear film. A high viscosity oil-based coating can act as a competing liquid that adheres better than the tear layer/water and is more energetically favorable to cover the hydrophobic coating material. For example, before inserting the device 10, the user can spread the high viscosity oil-based coating over the optically transparent hydrophobic surface of the device.

The optoelectronic components 22 can be positioned on the mounting substrate 14 of the device 10 to face towards the palpebral conjunctiva of the eyelid and can include one or more micro light emitting diodes (referred to as micro LED(s) 24) and a micro photodiode detector 26, which can be adjacent to the micro LED(s) 24. The micro LED(s) 24 can emit light toward the palpebral conjunctiva (through the infrared transparent encapsulation material 18 of the flexible casing 16) and the micro photodiode detector 26 can receive (detect) the light reflected from at least the palpebral conjunctiva, In one instance, the micro LED(s) 24 can emit short wave infrared light with wavelengths (or center wavelengths) in the range from 900 nm to 1700 nm toward the palpebral conjunctiva; and the photodetector can then detect the portion of the light reflected off the palpebral conjunctiva. As an example, the micro LEDs can include at least one micro LED that can emit short wave infrared light at wavelengths (or center wavelengths) from 1400 to 1500 nm and at least one other micro LED that can emit short wave infrared light at wavelengths (or center wavelengths) from 1300 to 1400 nm or from 1500-1700 nm. As an example, the micro LED(s) 24 can have a center wavelength of from 1525 nm to 1675 nm for measurement of a first overtone absorption peak of glucose. And another of the micro LED(s) 24 can have a center wavelength of from 1425 nm to 1575 nm (such as 1450 nm) for measurement of primarily water to remove any complicating signal components caused by the presence of water (from tears) between the device and the palpebral conjunctiva. For example, the full width half max of micro LED(s) 24 can be in the range of +/−60 nm. For instance, the micro LED(s) 24 can be commercially available short wave infrared (SWIR) micro LEDs in bare die forms that can be typically <100 μm thick and <250 μm in cross sectional length and width dimensions). At the wavelengths (or center wavelengths) discussed above glucose can be detected in the blood of the blood vessels in the palpebral conjunctiva and the interrupting signal from residual tears between the device 10 and the palpebral conjunctiva can be partially or fully removed. Other wavelengths (or center wavelengths) can be tuned (e.g., using different micro LED(s)) for detecting other chemical analytes in blood. The micro photodiode detector 26 can be positioned adjacent to the micro LED(s) 24 and configured to detect the reflected light from at least the palpebral conjunctiva of the patient in response to the short wave infrared light emitted by the micro LED(s) 24 interacting with at least the palpebral conjunctiva.

While water and lipid tissue are present in the epithelium and adenoid layers, water and lipid provide tissue in the layers provide constant background signals and do not have peaks which directly interfere with the glucose first absorption overtone. However, variations in the tear film thickness could result in some transient variations in absorption and thus would reduce the accuracy of the glucose measurement. Thus, suppressing the tear film variation as discussed using a material having hydrophobic surface properties and an oil-based viscous coating can greatly enhance the resolution of the analyte measurement.

Other ways of improving measurement resolution include using micro LEDS with narrower wavelength specificity as some LEDs can emit a wide wavelength spectrum with a full width half max of up to +/−60 nm. To further improve detection sensitivity, the detectors and/or emitters can be coated with different ultra-thin dielectric filters (directly or positioned underneath another thin filter element) to fine tune the detected spectral wavelengths by blocking out any unwanted signal which can degrade the measurement. These ultra-thin filter elements can be on the order of a few hundred microns thick and can be made to collect signals over a desired passband.

Other wavelengths (or center wavelengths) can be tuned (e.g., using different micro LED(s)) for detecting other chemical analytes in blood. The micro photodiode detector 26 can be positioned adjacent to the micro LED(s) 24 and configured to detect the reflected light from at least the palpebral conjunctiva of the patient in response to the short wave infrared light emitted by the micro LED(s) 24 interacting with at least the palpebral conjunctiva.

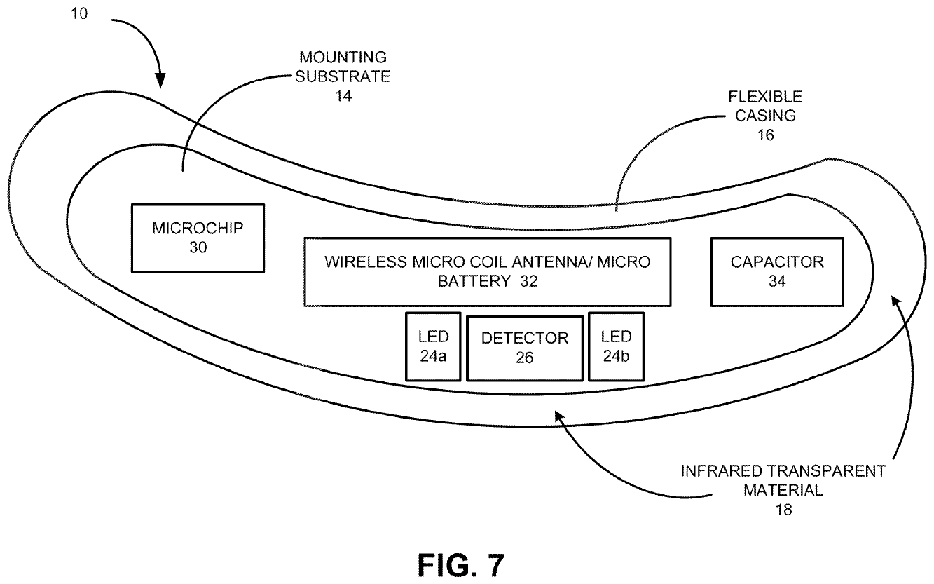

FIG. 7 shows an example device 10, with example circuit components of the microcircuit 20 are shown with an example configuration of the optoelectronic components 22. It should be understood that the configuration of components shown in FIG. 7 is for example purposes only and any practical configurations can be used. Additionally, electrical connections are not shown between components but should be understood as present. The microcircuit(s) 20 can include at least at least a microchip 20 (including, for example, a power scavenging NFC ASIC microchip), a wireless micro coil antenna and/or micro battery 32 (including, for example, an angled surface mount technology (SMT) microcoil inductor train, also referred to as a wireless micro-coil antenna), and a matching capacitor 34. For example, the wireless micro-coil antenna (which may include a ferrite core) can engage in NFC with a remote device and can harvest wireless energy via the NFC to power at least the optoelectronic components. A wireless micro-coil antenna can have a much smaller substrate area than a thin film coil antenna and can therefore allow for a device form factor that can be small enough to comfortably sit beside the eye in the palpebral conjunctiva.

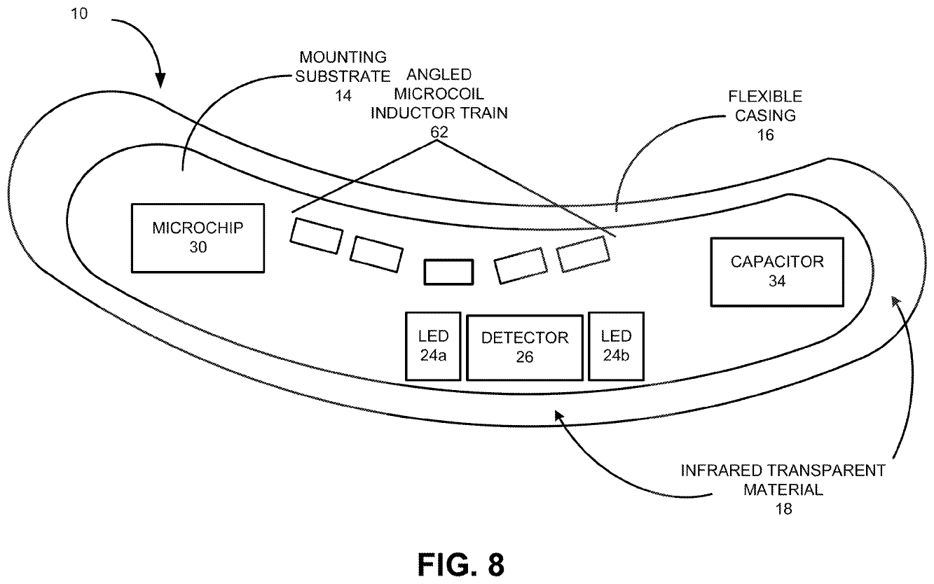

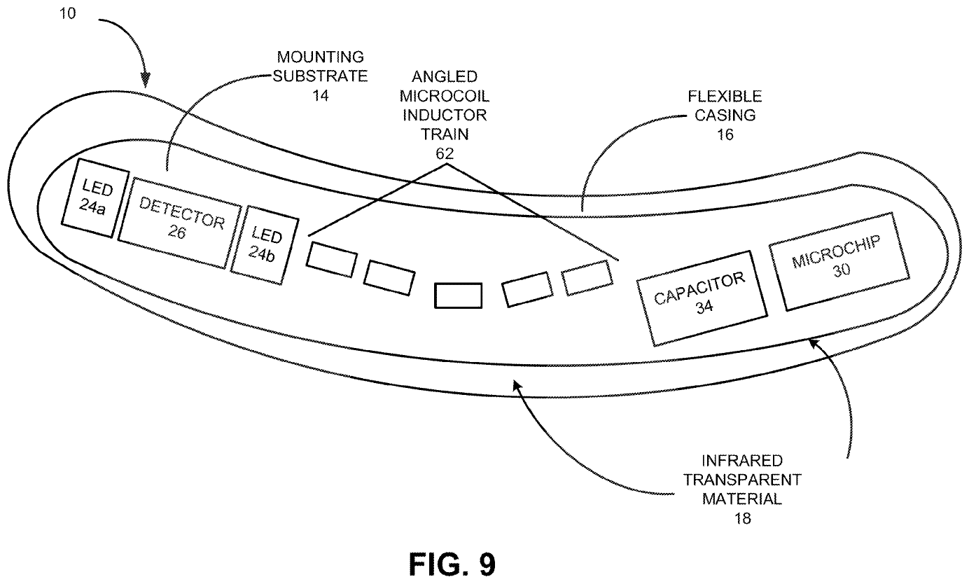

The wireless micro-coil antenna can be, for example, a line microcoil SMT array (as shown in FIGS. 8 and 9 as angled micro-coil inductor train 62) with shared flux for a total distributed inductance of 1 μH-1.5 μH with a 30-100 pF SMT cap matching capacitor 34 for energy harvested NFC. The onboard microchip 20 can rectify the wireless energy to power the optoelectronic components (e.g., the micro LEDs 24a and b and the detector 26 (the micro photodiode detector)) and can modulate a signal that the antenna communicates via NFC to the remote device (e.g., for energy harvesting and/or data communication). The wireless micro-coil antenna can include micro coil inductors with a high permeability core, such as at least one micro-coil wrapped around ferrite, (for example, ferrite microcoil wire inductors having an inductance of about 0.2 μH-0.3 μH). It should be understood that the wireless micro-coil antenna can be located as illustrated or in any position on and/or in the mounting substrate 14 and in communication with micro LEDs 24a and b and the micro detector 26 (e.g., the optoelectronic components). Such a magnetic ferrite-based inductor can also assist in the removal of the device from the underlid position. For example, a permanent magnet removal tool (e.g., a soft, small permanent magnet removal tool) sized and shaped to provide attractive forces to the magnetic ferrite-based inductor can magnetically attract the magnetic elements in the device 10 and when moved can force the device to slip out from underneath the lower lid without the need for a person (e.g., the user, medical professional, helper, or the like) to manually remove the device with one or more fingers. It should also be noted that the device 10 can include other elements (not shown) such as a micro temperature sensor.

In the example shown in FIG. 7, the optoelectronic components can include at least two micro LEDs 24a and 24b and a shared micro photodiode detector (e.g., detector 26). As an example, one of the micro LEDs 24a can be configured to emit short wave infrared light at center wavelengths from 1400 nm to 1500 nm (such as 1450 nm) and the other of the micro LEDs can be configured to emit short wave infrared light at wavelengths (or center wavelengths) from 1300 nm to 1400 nm (such as 1350 nm) or from 1500 nm-1700 nm (such as 1600 nm) The at least two micro LEDs 24a and 24b can be positioned adjacent or near one another and the shared micro photodiode detector (detector 26) can be positioned adjacent to (e.g., between as shown, above, below, beside one, or the like) the at least two micro LEDs 24a and 24b. The shared micro photodiode detector (detector 26) can be a InGaAs (indium gallium arsenide) detector. Different possible positionings of the at least two micro LEDs 24a and 24b and the detector 26 with an angled micro-coil inductor train 62 are shown in FIGS. 8 and 9 (although it should be understood that other positions are possible).

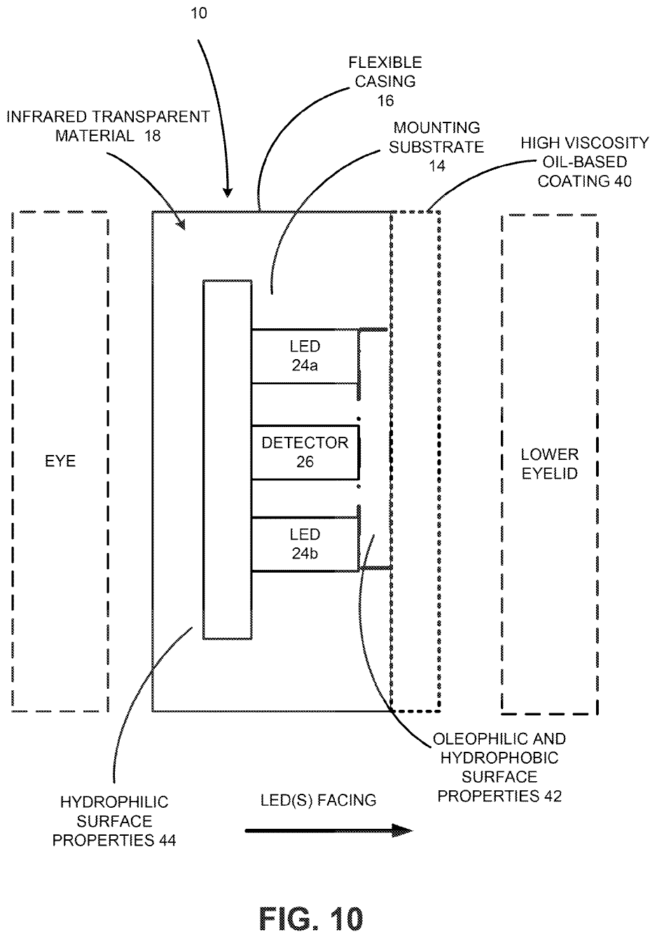

FIG. 10 is a diagram showing a side-view of a portion of the device 10 simplified to show only the optoelectronic components 22 (the other components from FIGS. 6-9 are not shown for simplicity's sake only) relative to the eye and the lower eyelid. The device 10 is shown in rectangles for simplicity, but it should be understood it has the curved construction and rounded edges described above. The position of the eye and the eyelid are also shown as rectangles for simplicity. As shown in FIG. 10, the device is positioned such that the optoelectronic components, the micro LEDs 24a and 24b and the micro photodiode detector (detector 26) face towards the eyelid (and the palpebral conjunctiva not shown). At least some of the exterior flexible casing 16 (e.g., on the side facing the lower eyelid) can be covered with the high viscosity oil-based coating 40. The high viscosity oil-based coating 40 can be, for example, an FDA accepted monograph formulation comprising some combination of medical grade mineral oil and medical grade white petrolatum used in overnight dry eye tear ointments. The infrared transparent material 18 of the flexible casing 16 can include at least two portions, where at least a first position has hydrophobic surface properties 42, which is positioned between at least the optoelectronic components (e.g., LEDs 24a and 24b and detector 26) and at least a second portion has hydrophilic surface properties 44, which may be positioned around the remainder of the internal components of the device (e.g., mounting substrate 14, optoelectronic components, microcircuitry, etc.). The portion of the infrared transparent material 18 that has hydrophobic surface properties 42 is illustrated, as an example of where that portion may be positioned, with a box between the micro LED(s) 24a and 24b and the high viscosity oil-based coating 40. The portion of the infrared transparent material 18 with hydrophobic surface properties 42 can repel tears and other water heavy fluids, while attracting the high viscosity oil-based coating 40. The rest of the infrared transparent material 18 can have hydrophilic surface properties 44 and can attract the tears and other water heavy fluids. The hydrophobic surface properties 42 of the device 10 may not be enough on their own to entirely suppress the tear layer which has complex natural surfactants (mucins, proteins, or the like), which may still adhere to the hydrophobic coating allowing for a tear film. The high viscosity oil-based 40 coating can act as a competing liquid that adheres better than the tear layer/water and is more energetically favorable to cover the hydrophobic coating material. For example, before inserting the device 10, the user can spread the high viscosity oil-based coating over the optically transparent hydrophobic surface of the device.

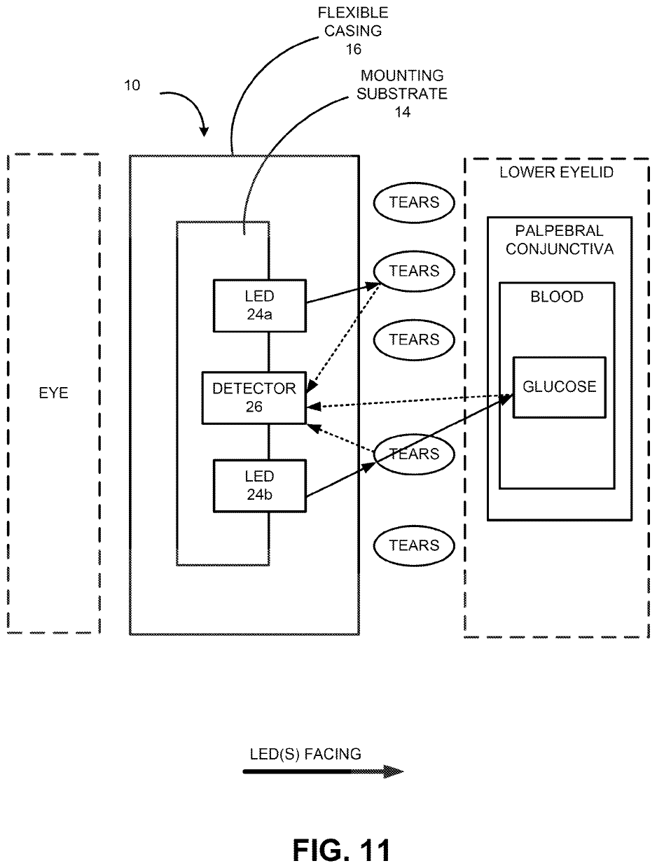

FIG. 11 is a diagram of a portion of FIG. 10 (without the infrared transparent material 18 (of the flexible casing 16) or the high viscosity oil-based coating 40 illustrated for simplicity) showing the operation of detecting glucose in a patient's blood in the presence of tears. It should be understood that the drawing is for ease of illustration and is not anatomically correct or to scale. The portion of the infrared transparent material 18 having hydrophobic surface properties 42 or the high viscosity oil-based coating 40 can repel/suppress the tears to an extent that may be greater than shown in FIG. 11 (e.g., such that substantially no tears, or a non-significant amount, are present between the device 10 and the lower eyelid). The palpebral conjunctiva is shown as a box in the lower eyelid box to express the connection between the two and can include numerous microvessels (represented as BLOOD) that can carry blood with a certain concentration of glucose. As previously mentioned, the palpebral conjunctiva is thin and highly vascularized with microvessels, generally derived from/supplied by the external carotid artery, many of which are surface blood vessels less than 100 μm from the surface of the eyelid. Thus, the light emitted from micro LEDs 24a and 24b and the reflected light received by the detector 26 can experience less interference and/or complicating other signals. As shown, while the device 10 is configured to minimize the presence of tears between the device and the palpebral conjunctiva of the lower eyelid, some small amount of tear film and water in the basil cell collagen like layers may remain and cause a complicating signal when light emitted by the micro LEDs 24a and 24b. For example, the complicating signal can be seen as a small parasitic diffusive scattering even rather than a diffusive reflection off of the chemical analyte (e.g., glucose in this example) in the blood of the palpebral conjunctiva.

The light sources, represented as micro LEDs 24a and 24b, can include micro LED 24a, which can be an analyte detection micro LED can emit a light having a center wavelength from 1525 to 1675 nm to reflect off glucose in blood in the palpebral conjunctiva of the patient, and micro LED 24b, a water detection reference LED can emit a light having a center wavelength from 1400-1500 nm to reflect off water in tears of the patient. The micro photodiode detector (e.g., detector 26) can receive reflected light from the chemical analyte in the blood (e.g., glucose) in the palpebral conjunctiva of the patient and reflected light from any water in the tears of the patient. The primary signal of the reflected light, received by the detector 26, from light emitted by the analyte detection micro LED can be from the chemical analyte (e.g., glucose) but may include secondary contributions from water or other interference. The primary signal of the reflected light, received by the detector 26, from light emitted by the water detection reference LED can be from water. A measurement of the chemical analyte (e.g., glucose) in the blood in the palpebral conjunctiva of the patient can be determined by subtracting a signal related to the reflected light from the water in tears of the patient from a signal primarily related to the reflected light from the chemical analyte in the blood in the palpebral conjunctiva of the patient. In one example, the signal detected based on light emitted from the micro LED 24a can be subtracted from the signal detect based on light emitted from the other micro LED 24b (e.g., by the micro photodetector 26) to remove the effects of water on the light signal used to opto-electronically monitor the glucose level in the patient's blood. Such a determination may be made by microcircuitry of the device 10 (e.g., including a microprocessor or other processing device not shown in this figure) and/or by the remote device 12 described previously (also not shown in this figure). Detection of glucose (or any other analyte) in the blood of the patient can be based on a predetermined schedule (e.g., every 5 minutes, every 10 minutes, every hour, every day, etc.) and/or can be manually induced via the remote device. Long term, uniform and accurate monitoring can therefore be achieved. Optionally, the remote device may alert (e.g., audibly, tactilely, and/or visually) the patient if a chemical analyte (e.g., glucose) reading at a given time is outside healthy limited (e.g., compared to one or more thresholds determined for the patient).

### IV. Method

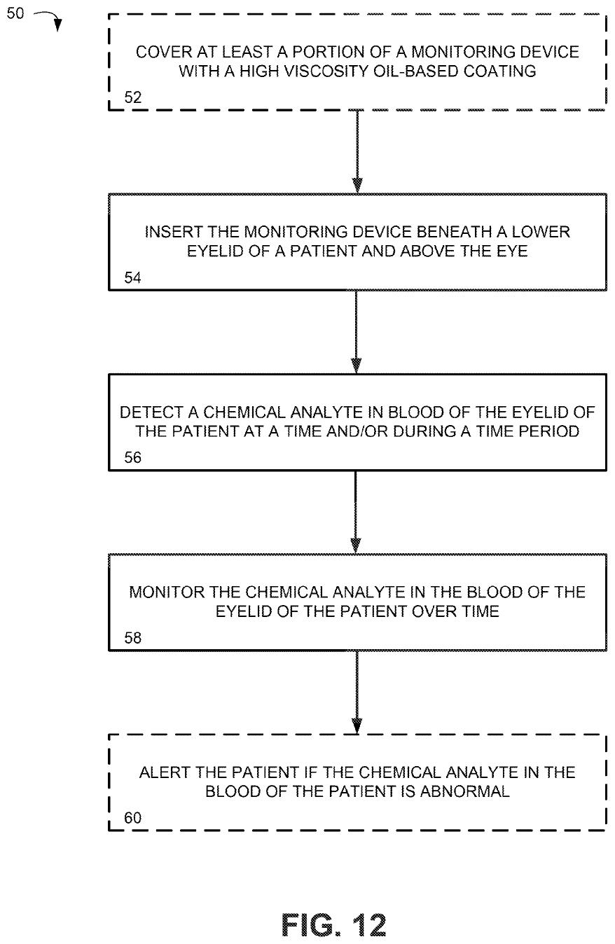

Another aspect of the present disclosure can include example methods 50, 70, 80, and 90 (shown in FIGS. 12, 13, 14, and 15) for monitoring a chemical analyte in a patient's blood using a non-invasive underlid detector (examples of which are shown as device 10 in FIGS. 1-11). The methods 50, 70, 80, and 90 are illustrated as process flow diagrams with flowchart illustrations that can be implemented as device 10 as shown in FIGS. 1-11.

For purposes of simplicity, the methods 50, 70, 80, and 90 are shown and described as being executed serially; however, it is to be understood and appreciated that the present disclosure is not limited by the illustrated order as some steps could occur in different orders and/or concurrently with other steps shown and described herein. Moreover, not all illustrated aspects may be required to implement the methods 50, 70, 80, and 90. It should be noted that one or more steps of the methods 50, 70, 80, and 90 can be executed by a hardware processor.

FIG. 12 is a process flow diagram of an example method 50 for monitoring a chemical analyte in a patient's blood using a non-invasive underlid detector (e.g., device 10). At 52, optionally, a surface of at least a portion of the monitoring device (e.g., device 10) can be covered with a high viscosity oil-based coating. The portion of the monitoring device covered with the high viscosity oil-based coating can be at least a portion of the flexible casing of the monitoring device (e.g., device 10) at least on the side facing the lower eyelid. The high viscosity oil-based coating can lubricate the device and/or keep at least some tear fluid away from the area between any optoelectronics (e.g., micro LED(s) 24 and micro photodetector 26) and the lower eyelid.

At 54, the monitoring device (e.g., device 10) can be inserted into the area beneath a lower eyelid of a patient and above the eye of the patient (e.g., see simplistic illustration of FIG. 3). The monitoring device can include optoelectronic components (e.g., one or more micro LEDs 24 and a micro photodiode detector 26) and one or more microcircuits (e.g., microcircuit(s) 20 that include at least a microchip 30, a wireless microcoil antenna/micro battery 32, and a matching capacitor 34) on a substrate (e.g., mounting substrate 14). The substrate, the one or more microcircuits, and the optoelectronic components can be encapsulated within a flexible casing (e.g., casing 16) that includes an infrared transparent material (e.g., infrared transparent material 18). The infrared transparent material can have at least two portions, where at least a first portion has hydrophobic surface properties, which can be positioned between at least the optoelectronic components of the device and the lower eyelid of the patient, and at least a second portion that has hydrophilic surface properties, which can be positioned around the remainder of the device. The portion of the infrared transparent material with hydrophobic surface properties can repel tears and other water heavy fluids and can also attract the high-viscosity oil-based coating. The rest of the infrared transparent material with hydrophilic surface properties can attract the tears and other water heavy fluids. The hydrophobic surface properties of the device may not be enough on their own to entirely suppress the tear layer which has complex natural surfactants (mucins, proteins, or the like), which may still adhere to the hydrophobic coating allowing for a tear film build up. The high viscosity oil-based coating can act as a competing liquid that adheres better than the tear layer/water and is more energetically favorable to cover the hydrophobic coating material. As such, prior to inserting the device the user can spread the high viscosity oil-based coating over the optically transparent hydrophobicsurface of the device.

At 56, a chemical analyte can be detected in blood of the eyelid of the patient at a time and/or during a time period (e.g., a predetermined time at a given rate or a manually selected time). The chemical analyte can be, for example, glucose. Other example chemical analytes include, but are not limited to cholesterol, potassium, iron, inorganic phosphorus, magnesium, lactate dehydrogenase (LDH), lactate, insulin, parathyroid hormone, osteocalcin, adrenocorticotropic hormone, or the like. The detection can be done by the optoelectronic components where the micro LEDs emit light towards the palpebral conjunctiva of the lower eyelid and the micro photodetector detects the reflected light. The light emitted by the micro LEDs can be shortwave infrared light with a wavelength (or center wavelength) from 1300 nm to 1700 nm. In some instances, the wavelength (or center wavelength) can be from 1300-1400, 1400-1500, 1600-1700 nm, or the like. For example, one micro LED can emit a light having a center wavelength from 1525 to 1675 nm to primarily reflect off glucose in blood in the palpebral conjunctiva of the patient, and another micro LED can emit a light having a center wavelength from 1400-1500 nm to primarily reflect off water in tears of the patient. At 58, the chemical analyte in the blood of the eyelid can be monitored over time. For example, the monitoring can occur until the patient removes the device (e.g., device 10) in time periods from hours to days to weeks or months. The monitoring may be done by the device itself, using a microprocessor or other processing component, and/or by a wireless device associated with the patient and in communication with the device. Optionally, at 60, the wireless device associated with the patient can alert the patient if the chemical analyte in the blood of the patient is abnormal. In all instances, the wireless device and the device (e.g., as shown in FIG. 1) can communicate via at least near field communication, where the communication can include data communication and/or power communication.

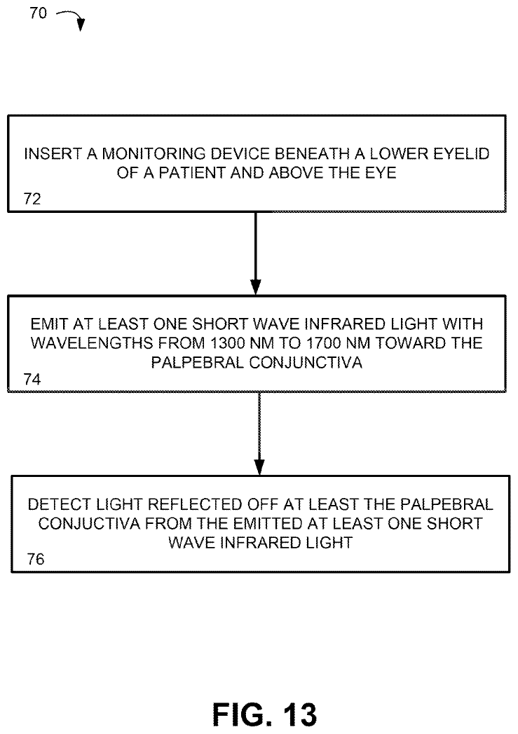

FIG. 13 is a process flow diagram of an example method 70 for using a non-invasive underlid detector (e.g., detector 10 with optoelectronic components 22). At 72, a monitoring device can be inserted beneath a lower eyelid of a patient and above the eye (as shown in FIG. 3, for example) with the optoelectronic components facing towards the lower eyelid. At 74, at least one short wave infrared light can be emitted (e.g., by at least one micro LED 24) with one or more wavelengths from 1300 to 1700 nm toward the palpebral conjunctiva of the patient's lower eyelid. The light can hit the palpebral conjunctiva (and more accurately blood vessels running through the palpebral conjunctiva) and be reflected, at least primarily, off of a given chemical analyte in blood in the blood vessels running through the palpebral conjunctiva. The center wavelength and spread of wavelength can be chosen based on the given chemical analyte to be detected. At 76, light reflected off at least the palpebral conjunctiva (or more accurately, the given chemical analyte in the blood in the blood vessels running through the palpebral conjunctiva) can be detected (e.g., by a micro photodiode detector 26). The concentration of the analyte of interest can be determined based on the detected reflected light. In some instances, more than one light (e.g., having different center wavelengths and/or spreads) can be emitted from more than one micro LED and the micro photodiode detector can detect the reflected light from one or more chemical analytes in the blood or interfering constructs (e.g., tears, skin, fat, or the like), and detected.

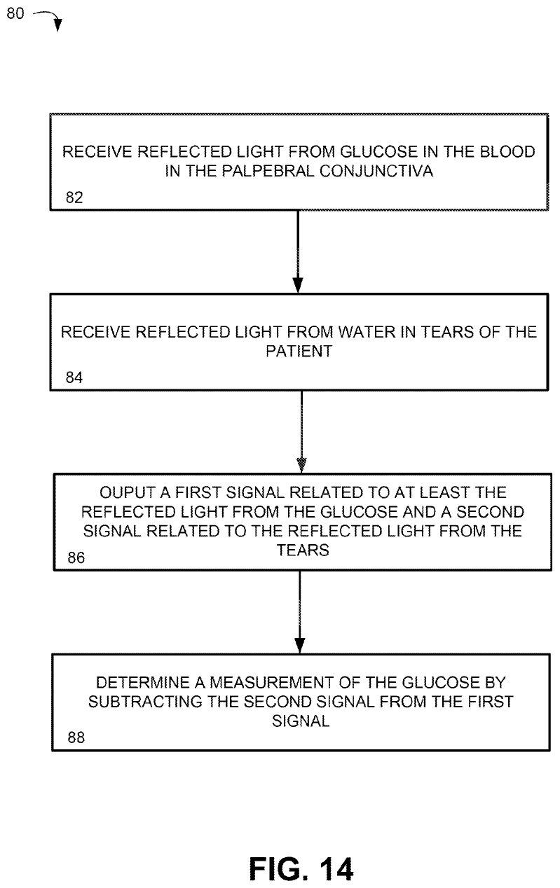

FIG. 14 is a process flow diagram of an example method 80 for monitoring a glucose concentration in a patient's blood using a non-invasive underlid detector (e.g., detector 10). At 82, the reflected light from glucose in the blood of the palpebral conjunctiva can be received (e.g., by micro photodiode detector 26). At 84, reflected light from water in tears of the patient can be received (e.g., by micro photodiode detector 26). At 86, a first signal can be output (e.g., by micro photodiode detector 26) related to at least the reflected light from the glucose and a second signal related to the reflected light from the tears. At 88, a measurement of the glucose can be determined (e.g., by a device 10 and/or a remote device 12 comprising a processor) by subtracting the second signal from the first signal. The measurement can be reported to the patient and/or a medical professional associated with the patient. When the measurement of glucose is beyond a preset threshold or other preset value, the report of the measurement can be accompanied by an alarm (e.g., an audio alarm and/or a visual alarm).

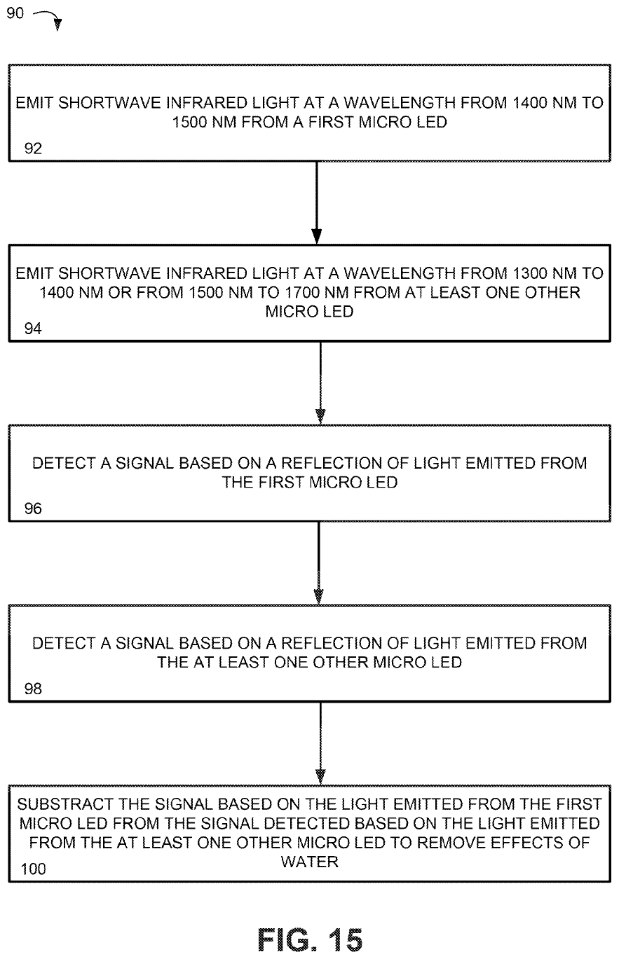

FIG. 15 is a process flow diagram of an example method 90 for monitoring a glucose concentration in a patient's blood using a non-invasive underlid detector (e.g., detector 10) in the presence of another fluid. At 92, shortwave infrared light can be emitted (e.g., by a first micro LED 24a) with a wavelength (or center wavelength) from 1400 to 1500 nm. Such a light can primarily reflect off water in any tears between the device and the palpebral conjunctiva of the lower eyelid. At 94, another shortwave infrared light can be emitted (e.g., by other micro LED 24b) with a wavelength (or center wavelength) from 1300 to 1400 nm or from 1500-1700 nm. Such a light can primarily reflect off a chemical analyte, such as glucose in blood in the palpebral conjunctiva of the lower eyelid of the patient. At 96, a signal can be detected (e.g., by a micro photodiode detector 26) based on a reflection of the first light emitted from the first micro LED. At 98, another signal can be detected (e.g., by micro photodiode detector 26) based on the light emitted from the at least one other micro LED. At 100, the signal based on the reflection of the first light emitted from the first micro LED can be subtracted from the signal based on the reflection of the light emitted from the at least one other micro LED (e.g., by a device 10 and/or a remote device 12 comprising a processor) to remove the effects of water on the signal of the one or more chemical analyte being monitored.

From the above description, those skilled in the art will perceive improvements, changes and modifications. Such improvements, changes and modifications are within the skill of one in the art and are intended to be covered by the appended claims.

1. A device configured to monitor a chemical analyte in a patient's blood, the device comprising:
a mounting substrate configured to hold at least one microcircuit with wireless communication and a wireless power source and optoelectronic components, wherein the optoelectronic components comprise:
one or more light sources configured to emit short wave infrared light with wavelengths from 900 nm to 1700 nm toward a palpebral conjunctiva of the patient; and
one or more light sensors configured to detect light from the palpebral conjunctiva of the patient; and
a flexible casing configured to fit between the lower eyelid and an eyeball of the patient, wherein the casing encapsulates the mounting substrate and the optoelectronic components, and wherein the casing comprises an infrared-transparent material,
wherein a portion of the infrared-transparent material above at least the one or more light sources and one or more light sensors comprises hydrophobic surface properties,
wherein the device is configured to be inserted between a lower eyelid and eye of the patient.

2. The device of claim 1, wherein the chemical analyte in blood is glucose.

3. The device of claim 2, wherein the one or more light sources configured to emit short wave infrared light having a center wavelength of from 1525 nm to 1675 nm for measurement of a first overtone absorption peak of the glucose.

4. The device of claim 1, wherein at least a portion of the casing above the one or more light sources and the one or more light sensors is covered with a high viscosity oil-based coating.

5. The device of claim 4, wherein the high viscosity oil-based coating comprises a medical grade mineral oil and/or a medical grade white petrolatum.

6. The device of claim 1, wherein at least another portion of the infrared transparent material comprises hydrophilic surface properties.

7. The device of claim 1, wherein the mounting substrate is an active flex printed circuit board (PCB) that is configured to curve with a radius of curvature the eyeball of the patient.

8. The device of claim 1, wherein the one or more micro LEDs comprises a micro LED that emits short wave infrared light at a wavelength from 1400 nm to 1500 nm and at least one other micro LED configured to emit short wave infrared light at one or more other wavelengths from 1300 nm to 1400 nm or from 1500 nm to 1700 nm.

9. The device of claim 8, wherein a signal detected based on light emitted from the micro LED is subtracted from the signal detected based on light emitted from the at least one other micro LED to remove effects of water.

10. The device of claim 1, wherein the one or more micro LED comprises:
an analyte detection micro LED configured to emit a light having a center wavelength from 1525 to 1675 nm to reflect off glucose in blood in the palpebral conjunctiva of the patient; and
a water detection reference LED configured to emit a light having a center wavelength from 1400-1500 nm to reflect off water in tears of the patient.

11. The device of claim 10, wherein the micro photodiode detector is configured to receive reflected light from the chemical analyte in the blood in the palpebral conjunctiva of the patient and reflected light from the water in the tears of the patient,
wherein a measurement of the chemical analyte in the blood in the palpebral conjunctiva of the patient is determined by subtracting a signal related to the reflected light from the water in tears of the patient from a signal related to the reflected light from the chemical analyte in the blood in the palpebral conjunctiva of the patient.

12. The device of claim 1, further comprising:
a wireless antenna enabling wireless power transfer and/or wireless communication with a remote transponder.

13. A method for monitoring a chemical analyte in blood of a patient's palpebral conjunctiva, the method comprising:
inserting a device beneath a lower eyelid of the patient and above an eye,
wherein the device comprises:
a mounting substrate configured to hold a microcircuit and to mount optoelectronic components, wherein the optoelectronic components comprise:
one or more micro light emitting diodes (LEDs); and
a micro photodiode detector, adjacent to the one or more LEDs,
wherein the mounting substrate and the optoelectronic components are covered and protected by an infrared transparent encapsulation material,
wherein a surface of the infrared transparent encapsulation material has hydrophobic surface properties in a region above the one or more micro LEDs and the photodiode detector;
emitting, by the one or more micro LEDs, short wave infrared light with wavelengths from 900 nm to 1700 nm toward the palpebral conjunctiva; and
detecting, by the photodetector, light reflected off the palpebral conjunctiva after receiving the short wave infrared light emitted by the one or more micro LEDs,
wherein the chemical analyte in the blood is monitored based on the detecting.

14. The method of claim 13, wherein the chemical analyte in blood is glucose.

15. The method of claim 14, wherein the detecting further comprises:
receiving, by the photodetector, reflected light from the chemical analyte in the blood in the palpebral conjunctiva of the patient and reflected light from water in tears of the patient;
outputting, by the photodetector, a first signal related to the reflected light from the chemical analyte in the blood in the palpebral conjunctiva of the patient and a second signal reflected light from water in tears of the patient; and
determining, by an external device and/or the microcircuit, a measurement of the chemical analyte in the blood in the palpebral conjunctiva of the patient by subtracting the second signal from the first signal.

16. The method of claim 13, further comprising: before the inserting, covering at least a portion of the infrared transparent encapsulation material having the surface with hydrophobic surface properties with a high viscosity oil-based coating.
